(19) United States
(12) Patent Application Publication
(10) Pub. No.: US 2021/0212947 A1
(43) Pub. Date: Jul. 15, 2021

(54) Docetaxel palmitate liposome and preparation method thereof

(71) Applicant: SHANGHAI WEI ER BIOPHARMACEUTICAL TECH CO., LTD., Shanghai (CN)

(72) Inventors: Jianming CHEN, Shanghai (CN); Baoan GAO, Shanghai (CN); Youfa XU, Shanghai (CN); Xin WU, Shanghai (CN); Yamin SHI, Shanghai (CN); Lang YAN, Shanghai (CN); Zhiqin FU, Shanghai (CN); Xiaoping LI, Shanghai (CN)

(21) Appl. No.: 17/056,139

(22) PCT Filed: Apr. 26, 2019
(86) PCT No.: PCT/CN2019/084543

§ 371 Date: Nov. 17, 2020

(30) Foreign Application Priority Data
May 18, 2018 (CN) 201810480115.3

(51) Int. Cl. A61K 9/127; A61K 31/337; A61K 9/00; A61K 47/28; A61K 47/24; A61K 47/18; A61K 47/12; A61K 47/26

(52) U.S. Cl. CPC A61K 9/1273; A61K 31/337; A61K 9/0019; A61K 9/1277; A61K 47/26; A61K 47/24; A61K 47/183; A61K 47/12; A61K 47/28

(57) ABSTRACT

The present invention relates to the technical field of medicine, which provides a liposomal docetaxel palmitate formulation and a method for preparing the said formulation. The liposomal docetaxel palmitate formulation contains docetaxel palmitate as the main drug, a chelating agent, lecithin and DSPE-PEG2000. It is characterized in that docetaxel is prepared into a docetaxel palmitate lipophilic prodrug, thus overcoming the defect that docetaxel cannot be processed into liposomes due to poor hydrophilicity and hydrophobicity. The liposome prescription of this invention contains a chelating agent with a substantive effect that can prolong the action time of the drug in the body, improve the anti-tumor effect and be prepared smoothly. Therefore, the chelating agent in the formulation is the core technical feature of the present invention. The purpose of the present invention is to develop a more efficient docetaxel palmitate liposome with no solubilizer so that it can be prepared more easily

### TECHNICAL FIELD

The invention relates to the technical field of medicine, in particular to a docetaxel palmitate liposome and a preparation method thereof.

### BACKGROUND TECHNOLOGY

Docetaxel (DTX), also known as taxotere, is a taxane anti-tumor drug modified with 10-deacetylbaccatin III as the core skeleton. Its chemical structure is shown in FIG. 1. As shown, the chemical name is: [2aR-(2aα,4β,4aβ,6β,9α(aR*,βS*),11a,12a,12aα,12bα)]-β-[[(1,1dimethylethyl (Oxy)carbonyl]amino]-α-hydroxyphenylpropionic acid [12b-acetoxy-12-benzoyloxy-2a,3,4,4a,5,6,9,10,11,12,12a,12b -Dodecahydro-4,6,11-trihydroxy-4a,8,13,13-tetramethyl-5-oxo-7,11-methylene-1H-cyclodecpentaeno [3,4 ]Benzo [1,2-b]oxetane-9-yl]ester, molecular formula C43H53NO14, molecular weight 807.88, insoluble in water, soluble in organic solvents such as ethanol, acetone, ether and benzene. The anti-tumor activity of docetaxel is 1.3-12 times that of paclitaxel, and the effect is definite. The FDA has approved it for the treatment of breast cancer, ovarian cancer, non-small cell lung cancer and pancreatic cancer. Docetaxel is one of the most valuable anticancer drugs found so far.

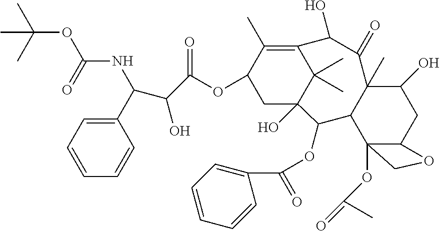 [SMILES: CC(=O)OC12COC1CC(O)C1(C)C(=O)C(O)C3=C(C)C(OC(=O)C(O)C(NC(=O)OC(C)(C)C)c4ccccc4)CC(O)(C(OC(=O)c4ccccc4)C21)C3(C)C]

### The Chemical Structure of Docetaxel

At present, the docetaxel preparation currently used in clinical practice is its injection form, Known as Docetaxel Injection, which is the only clinical dose form of docetaxel. The injection is composed of two parts: Tween 80 solution of docetaxel and 13% ethanol solution. When used, the 13% ethanol solution is mixed with Tween 80 solution of docetaxel, shaken well, and diluted with 5% glucose solution or normal saline (NS) before intravenous (IV) infusion. It can be seen that the clinical application process of the injection is cumbersome, inconvenient and prone to secondary contamination. Although the effect of Docetaxel Injection is significant, the adverse effects are particularly prominent. The primary adverse effect is bone marrow suppression toxicity, which has been clearly recorded in its instructions. When Docetaxel Injection is administered alone, the incidence of bone marrow suppression is as high as 76.4%. When it is used in combination with other chemotherapy drugs, the bone marrow suppression toxicity is more serious and the incidence is even higher, which seriously affects the chemotherapy process and weakens the treatment effect of patients (docetaxel Injection product manual; Zhu Kun, Zhou Can, Yan Rong, et al. Fluctuation of white blood cell count in patients with grade IV myelosuppression after conventional-dose chemotherapy with docetaxel treatment strategies [J].Modern Oncology, 2012, 20(1):000159-161.; Cao Jianwei, GengMingfei, Zhu Dongshan, et al. The characteristics and countermeasures of bone marrow suppression in patients with esophageal cancer caused by docetaxel chemotherapy[J].Chinese National Health and Medical Sciences, 2016, 28(11):10-11.; Sha Hongyu, Zheng Wenwen, Guo Chenyu, et al. Analysis of adverse reaction reports caused by docetaxel[J].Chinese Journal of Hospital Pharmacy,2015,35(6):536-539.). In addition, Tween 80 in Docetaxel Injection has hemolytic and allergic properties, which may bring about serious safety hazards to clinical medication and severely limit the anti-tumor efficacy of docetaxel. Therefore, there is an urgent need to develop a non-solubilizing docetaxel injection with higher therapeutic efficacy and lower bone marrow suppression toxicity for clinical use, as well lay a foundation for in-depth research and application of docetaxel.

Given the shortcomings of Docetaxel Injection currently available, many efforts have been attempted to develop new docetaxel preparations by preparing docetaxel into liposomes, micelles, nanoparticles and other nano-encapsulated preparations (Cheng Shucang, Pang Xin, Zhai Guangxi. Duo The research progress of nepaclitaxel nano-preparation[J]. Pharmaceutical Research, 2013,32(1):45-48.), but unfortunately no new docetaxel nano-preparation has been commercially available so far. From the perspective of reducing the toxicity and improving the curative effect, liposomes have been widely studied and applied as carriers of anti-tumor drugs. In particular, liposomes of adriamycin and irinotecan, which have been used clinically, have proved remarkably effective in reducing the toxicity and improving the curative effect. Therefore, most researchers in this field are also planning to prepare docetaxel into liposomes to achieve the goal of safety and efficiency. A docetaxel liposome with a remarkably effective drug loading amount of 0.75 mg/mL has been reported in the literature, but as it has poor stability and cannot be stored for a long time, it cannot be used clinically (Immordino ML, Brusa P, Arpicco S, et al. Preparation, characterization, cytotoxicity and pharmacokinetics of liposomes containing docetaxel[J]. Journal of Controlled Release, 2003, 91(3):417.). The highest drug loading of docetaxel liposomes reported in the existing literature is 1 mg/mL. But these docetaxel liposomes are not conducive to industrializatio due to poor stability and the complex preparation process (Patel K, Doddapaneni R, Chowdhury N, et al. Tumor stromal disrupting agent enhances the anticancer efficacy of docetaxel loaded PEGylated liposomes in lung cancer[J]. Nanomedicine, 2016, 11(11):1377-1392.). A Chinese patent (Patent No. CN101584663A) reports a new type of docetaxel liposome for injection and the preparation method of emulsification and volatility. But the preparation process of the reported Docetaxel liposomes is complex and uncontrollable, and the prescription contains sodium cholesterol sulfate and sodium dodecylbenzene sulfonate plasma type solubilizers, which have strong hemolytic properties (Cui Fude. Pharmaceutical Studies [M]. Beijing: People's Medical Publishing House, 2011: 42). Another Chinese patent (Patent No. CN103830181A) discloses a freeze-dried docetaxel liposome and the preparation method by adding hemolytic cyclodextrin to improve the water solubility of docetaxel, increase the encapsulation efficiency, and improve the stability of liposomes. Even so, the drug loading of liposomes is only 0.5 mg/mL, which cannot meet the requirements of clinical medication. Still another Chinese patent (Patent No. CN102379849A) provides a pH-sensitive docetaxel liposome and the preparation method, but the drug loading is still low and the liposome particle size is too large. The above examples indicate the poor feasibility of directly preparing docetaxel into liposomes, mainly due to the poor fat solubility of docetaxel and the mismatch of compatibility with lipid materials. Plastids have a series of problems such as low drug loading, low encapsulation efficiency, and poor stability. All in all, the druggability of docetaxel liposomes is very poor. Therefore, it is particularly important to develop a docetaxel liposome with high efficiency, low toxicity, stable quality and a simple preparation process, which would lay a solid foundation for the basic research and clinical application of docetaxel in the anti-tumor field.

### SUMMARY OF THE INVENTION

The disclosure provides a docetaxel palmitate liposome formulation. In order to solve the problem of poor fat solubility of docetaxel and poor drug ability of liposomes, the inventors plan to improve the fat solubility of docetaxel through structural modification, where docetaxel and palmitic acid are esterified to obtain a docetaxel fat-soluble prodrug known as docetaxel palmitate. The experiment of the present invention has demonstrated that liposomes prepared by a specific prescription process of docetaxel palmitate are excellent as a drug, with a drug loading as high as 10 mg/ml (Example 10), and the anti-tumor effect in mice is better than that of the commercial Docetaxel Injection (Example 19), so the present invention transforms the structure of docetaxel into docetaxel palmitate, which is one of the key technical features of the present invention to achieve the significant effects.

A Chinese patent CN201610301096.4 (Publication No. CN105853403A) describes a fat-soluble prodrug of paclitaxel known as paclitaxel palmitate and successfully developed its liposomes, which significantly improved the anti-tumor effect and safety in vivo. It is therefore not surprising to solve the problem of poor drugability of liposomes by synthesizing a fat-soluble prodrug to achieve the goal of high-efficiency and low-toxic effects in animals. In addition, docetaxel and paclitaxel are both taxane compounds. Theoretically speaking, docetaxel palmitate liposomes can achieve the expected purpose under the enlightenment of the above patents. In fact, the anti-tumor effect of docetaxel palmitate liposomes is indeed better than the commercially available Docetaxel Injection (Example 19). However, in the process of research, it was unexpectedly discovered that the introduction of a chelating agent in the prescription not only prolonged the action time of the drug in vivo (Example 20) but improved the anti-tumor effect simultaneously. Besides, the liposome-related quality indicators and other aspects were also improved after addition of the chelating agent. For instance, it narrowed the particle size distribution (PDS) and smoothed sterilization and filtration, both of which are beneficial to industrialized mass production and can greatly improve the applicability of the present invention (Example 21). Therefore, the chelating agent added to the docetaxel palmitate liposome is the unique technical characteristic of the present invention.

The metal atom or ion interacts with a ligand containing two or more coordinating atoms to form a chelate with a cyclic structure. This ligand substance that can form a chelate is called a chelating agent. In pharmaceutical preparations, chelating agents are widely used, but they are basically added to improve the chemical stability of the active ingredients. They are especially effective as antioxidants but can also be effective in preparations and prolong the time of action in the body. It is rare to show better results. No relevant report is available in the domestic and foreign literature. Whether the chelating agent in the liposome has an effect on the drug or has some binding to the liposome particle itself is temporarily unknown and further investigation is required in future. Out of curiosity, we carried out a series of comparative experiments and found that after adding a chelating agent to the docetaxel palmitate liposomes, the circulation of the drug in the body was significantly prolonged, and the area under the curve AUC∞ was also bigger than that without the chelating agent (Example 20). We know that drugs are slowly metabolized in the body and are not easily inactivated, so the efficacy of the drug is naturally improved. Therefore, if the present invention wants to achieve better substantive effects, it is far from enough to rely solely on the inspiration of the Chinese patent CN105853403A, because the invention provides a docetaxel palmitate liposome whose innovative technical feature is to contain a chelate and the mixture has also received substantial results.

The first objective of the present invention is to provide a docetaxel palmitate liposome.

The invention provides a docetaxel palmitate liposome, which uses docetaxel palmitate as the main medicine at a dose of 0.1-2% (weight volume percentage).

The present invention provides a docetaxel palmitate liposome, which takes docetaxel palmitate as the main drug and also includes a chelating agent. The dose of docetaxel palmitate and the chelating agent respectively is 0.1-2% and 0.001-1% (weight volume percentage) respectively.

The present invention provides a docetaxel palmitate liposome, which uses docetaxel palmitate as the main drug, and also includes a chelating agent, lecithin and DSPE-PEG2000. The dose of docetaxel palmitate, the chelating agent, lecithin and DSPE-PEG2000respectively is 0.1-2%, 0.001-1%, 1-10% and 0.05-1% (weight volume percentage) respectively.

The second objective of the present invention is to provide a docetaxel palmitate liposome, which is a lyophilized powder injection or a liposome solution for injection.

The third objective of the present invention is to provide a docetaxel palmitate prodrug, which uses docetaxel as the parent drug and links a molecule of palmitate with an ester bond, knowing that a prodrug formed by acid is a fat-soluble prodrug with good stability and strong functionality.

The structure of the docetaxel palmitate prodrug is as follows:

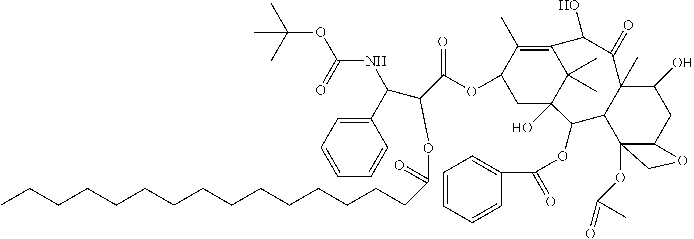 [SMILES: CCCCCCCCCCCCCCCC(=O)OC(C(=O)OC1CC2(O)C(OC(=O)c3ccccc3)C3C4(OC(C)=O)COC4CC(O)C3(C)C(=O)C(O)C(=C1C)C2(C)C)C(NC(=O)OC(C)(C)C)c1ccccc1]

The chemical structural formula of docetaxel palmitate

The docetaxel palmitate prodrug is characterized in that palmitic acid is connected to the 2′position of the side chain of docetaxel, and the preparation process is as follows: Docetaxel 10.00 g, palmitic acid 3.81g, 1-(3-Dimethylaminopropyl)-3-ethylcarbodiimide (EDC) 2.43 g and 4-dimethylaminopyridine (DMAP) 1.82 g are put in the reaction vessel and dissolved by addition of 50 mL anhydrous dichloromethane and stirring at room temperature for 4-24 h under the protection of nitrogen to obtain the reaction solution. The reaction solution was washed twice with 5% citric acid aqueous solution, and then with saturated sodium chloride solution once. Water dichloromethane is removed by rotary evaporation and pressure reduction, and docetaxel palmitate is finally separated and purified.

The reaction synthesis route diagram is as follows:

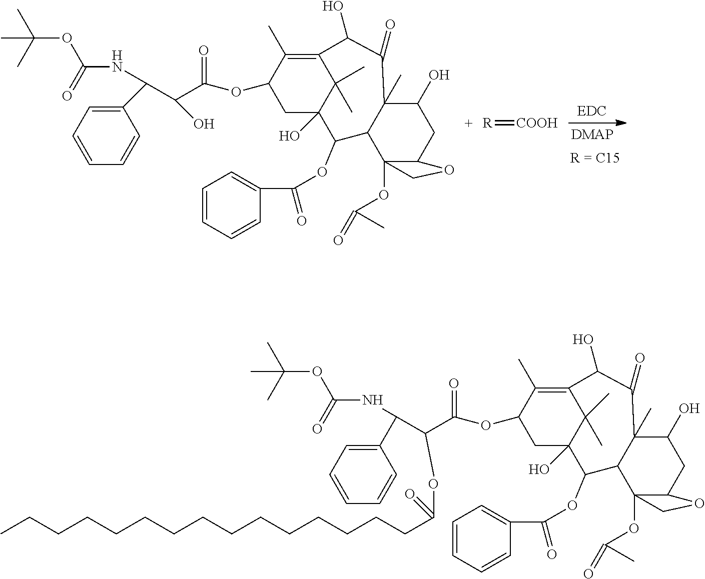

Synthetic route of docetaxel palmitate

The present invention provides a docetaxel palmitate liposome. The liposome is an injection containing a chelating agent; the injection that contains the chelating agent can also be an injection solution or a kind of freeze-dried powder injection. The chelating agent contained is the core technical feature of the present invention.

The said docetaxel palmitate liposome is specifically formulated by the following formula:
Docetaxel palmitate 0.1-1% (g/mL)
Lecithin 1-10% (g/mL)
DSPE-PEG₂₀₀₀ 0.05-1.0% (g/mL)
Cholesterol 0-1% (g/mL)
Chelating agent 0.001-1% (g/mL)
Lyophilized protective agent 0-40% (g/mL)
pH is adjusted to 3.5-9.0 using a pH adjuster.

The remaining is the water for injection.

When preparing the injection solution, the freeze-dried protective agent is 0;

When preparing the freeze-dried powder for injection, the freeze-dried protective agent is preferably 0.1-40% g/ml, and the said docetaxel palmitate liposome is specifically formulated by the following formula:
Docetaxel palmitate 0.1-0.8% (g/mL)
Lecithin 2-7% (g/mL)
DSPE-PEG₂₀₀₀ 0.1-0.8% (g/mL)
Cholesterol 0-0.6% (g/mL)
Chelating agent 0.005-0.8% (g/mL)
Lyophilized protective agent 5-35% (g/mL)
pH is adjusted to 3.5-8.0 using a pH adjuster.
The remaining is the water for injection.

Preferably, the said docetaxel palmitate liposome is specifically formulated by the following formula:
Docetaxel palmitate 0.2-0.7% (g/mL)
Lecithin 3-6% (g/mL)
DSPE-PEG₂₀₀₀ 0.2-0.7% (g/mL)
Cholesterol 0-0.5% (g/mL)
Chelating agent 0.01-0.5% (g/mL)
Lyophilized protective agent 10-30% (g/mL)
pH is adjusted to 3.5-7.0 using a pH adjuster.
The remaining is the water for injection.

Among them, the lecithin described in the above formula is selected from one or more of the following: high-purity egg yolk lecithin (EPCS), hydrogenated soybean lecithin (HSPC), dipalmitoyl phosphatidyl choline (DPPC), phosphatidyl choline, egg yolk lecithin, soybean lecithin, phosphatidylserine, myristical phosphatidylcholine, distearyl phosphatidylcholine, phosphatidylethanolamine and sphingomyelin; preferably high-purity egg yolk lecithin (EPCS), and hydrogenated soy lecithin (HSPC).

The chelating agent described in the appeal formula is selected from one or more the following: citric acid, disodium citrate, trisodium citrate, lactic acid, sodium lactate, malic acid, sodium malate, ethylenediaminetetraacetic acid, disodium ethylenediaminetetraacetic acid, and trisodium ethylenediaminetetraacetic acid, preferably one or a combination of two or more of citric acid, disodium citrate, trisodium citrate, lactic acid, and sodium lactate.

The freeze-drying protective agent described in the above formula is one or more of the following: trehalose, sucrose, maltose, lactose, mannitol, glucose, sorbitol, xylitol, erythritol and threonine, among which mannitol, trehalose or sucrose alone, and preferably their two or three-combination.

The pH adjusting agent in the above formula is one or more of the following: sodium hydroxide and hydrochloric acid.

The fourth objective of the present invention is to provide a method for preparing the said docetaxel palmitate liposome.

The method for preparing the docetaxel palmitate liposome is an injection method.

The said docetaxel palmitate liposome is prepared by the following steps: Weigh the prescription amount of docetaxel palmitate, cholesterol, phospholipid, DSPE-PEG2000, chelating agent, put them in an organic solvent for injection and dissolve them by heating at 25-70° C. to obtain the organic phase; heat a proper amount of water to 25-70° C. to obtain the water phase; pour the organic phase into the water phase under stirring and mix them well to obtain crude liposomes; emulsify the crude liposomes and place them under high pressure; perform homogenization and emulsification in a homogenizer, or place them in an extruder to extrude through extruded membranes with different pore diameters, or extrude after high-pressure homogenization to obtain a liposome solution; dry the protective agent, place it in the above liposome solution, dissolve it by stirring, and dilute it to the full volume with water for injection; adjust the pH value with a pH adjuster; finally, sterilize, pack and seal it through a 0.22 μm filter membrane to obtain so-called liposomes of liposome docetaxel palmitate. A powder form of docetaxel palmitate liposomes can also be prepared by lyophilization.

Wherein, the organic solvent for injection is selected from one or more of the following: propylene glycol, absolute ethanol and tert-butanol at the dose of 1-8% g/ml, among which absolute ethanol at a dose of 2-5% g/mL is more preferable.

The organic solvent for injection can be retained in liposomes, or removed by ultrafiltration or freeze-drying after the crude liposomes are emulsified. The said crude liposomes are emulsified, preferably by extrusion and emulsification methods, so that PDS of the liposomes obtained will be more uniform; the pore diameter of the extruded membrane is selected from one or more the following in descending order: 0.8 μm, 0.6 μm, 0.4 μm, 0.2 μm, 0.1 μm and 0.05 μm, among which 0.4 μm, 0.2 μm, 0.1 μm and 0.05 μm are more preferable.

The chelating agent can be dissolved in the oil phase, water phase, or liposome solution.

The freeze-dried protective agent is dissolved in the liposome solution, or the water phase.

An invented docetaxel palmitate liposome with a particle size of 50-150 nm.

The docetaxel palmitate liposome of the present invention contains a chelating agent, which is the core technical feature. Addition of the chelating agent enables docetaxel to act for a longer period of time and exert a better anti-tumor effect in vivo; in addition, it improves the preparation-related characteristics, all of which represent the substantial effect of the present invention.

### SPECIFIC IMPLEMENTATION MODES

The following is a detailed description about the present invention in conjunction with specific embodiments. It should be understood that the following examples are only used to illustrate the present invention and not to limit the scope of the present invention.

### Example 1

### Preparation of Docetaxel Palmitate Liposomes

[01] The organic phase was prepared with the prescription amount of 0.5 g docetaxel palmitate, 3 g high-purity egg yolk lecithin (EPCS), 0.3 g DSPE-PEG2000 and 4 g absolute ethanol. The mixture was dissolved by heating at 50° C. 0.05 g disodium ethylenediaminetetraacetic acid was put in 90 g water for injection and heated at 50° C. The resulting mixture was stirred to obtain an aqueous phase. The organic phase was injected into the water phase under stirring conditions to obtain crude liposomes, which were then placed in an extruder and separated through extruded membranes with a pore diameter of 0.4 μm, 0.1 μm and 0.05 μm to obtain liposome solution. The solution was diluted to 100 ml with water for injection, and the phase. The pH value was adjusted to 4.50 with hydrochloric acid. The liposomes were filtrated and sterilized through a 0.22 μm nylon syringe filter. The obtained filtrate was then separately packaged and cap-sealed to obtain docetaxel palmitate liposome solution with a mean particle size of 92.4 nm.

### Example 2

### Preparation of Docetaxel Palmitate Liposomes

[02] The organic phase was prepared with the prescription amount of 0.3 g docetaxel palmitate, 3 g high-purity egg yolk lecithin (EPCS), 0.2 g DSPE-PEG2000, 0.1 g citric acid and 4 g propylene glycol. The mixture was dissolved by heating at 60° C. 70 g water for injection was heated at 60° C. to obtain a water phase. The organic phase was injected into the water phase under stirring conditions to obtain crude liposomes, which were then placed in an extruder and sequentially passed through a nylon syringe filter of 0.4 μm, 0.2 μm, 0.1 μm and 0.05 μm to obtain liposome solution. 15 g saccharose and 5 g mannitol were dissolved in the liposome solution by stirring and diluted to 100 mL with water for injection. The pH value was adjusted to 5.50 with natrium hydroxydatum. The liposomes were filtrated and sterilized through a 0.22 μm nylon syringe filter, and the obtained filtrate was separately packaged, freeze-dried and cap-sealed to obtain a liposomal docetaxel palmitate freeze-dried powder with a mean particle size of 86.6 nm.

### Example 3

### Preparation of Docetaxel Palmitate Liposomes

[03] The organic phase was prepared with the prescription amount of 0.7 g docetaxel palmitate, 6 g high-purity egg yolk lecithin (EPCS), 0.5 g DSPE-PEG2000, 0.3 g citric acid and 6 g absolute ethanol. The mixture was dissolved by heating at 45° C. 65 g water for injection was heated to 45° C. to obtain a water phase. The organic phase was injected into the water phase under stirring conditions to obtain crude liposomes, which were then placed in an extruder and sequentially passed through a nylon syringe filter with a pore diameter of 0.6 μm, 0.4 μm and 0.1 μm to obtain liposome solution. 20 g trehalose was dissolved in the liposome solution by stirring and diluted to 100 mL with water for injection. The pH value was adjusted to 6.20 with natrium hydroxydatum. The liposome was filtrated and sterilized through a 0.22 μm nylon syringe filter. Then, the filtrate was separately packaged, freeze-dried and cap-sealed to obtain a liposomal docetaxel palmitate freeze-dried powder with a mean particle size of 120.7 nm.

### Example 4

### Preparation of Docetaxel Palmitate Liposomes

[04] The organic phase was prepared with the prescription amount of 0.3 g docetaxel palmitate, 5 g high-purity egg yolk lecithin (EPCS), 0.3 g DSPE-PEG2000, 0.1 g malic acid,0.2 g citric acid and 5 g absolute ethanol. The mixture was dissolved by heating at 65° C. 70 g water for injection was heated to 65° C. to obtain a water phase. The organic phase was injected into the water phase under stirring conditions to obtain crude liposomes, which were homogenized and emulsified by using a high pressure homogenizer, and then sequentially extruded with the extrusion film with a pore diameter of 0.1 μm and 0.05 μm to obtain liposome solution. 10 g saccharose and 5 gtrehalose were dissolved in the liposome solution by stirring and diluted to 100 ml with water for injection. The pH value was adjusted to 6.0 with natrium hydroxydatum. The liposome was filtrated and sterilized through a 0.22 μm nylon syringe filter, and the filtrate was then separately packaged, freeze-dried and cap-sealed to obtain a liposomal docetaxel palmitate freeze-dried powder with a mean particle size of 50.36 nm.

### Example 5

### Preparation of Docetaxel Palmitate Liposomes

[05] The organic phase was prepared with the prescription amount of 0.2 g docetaxel palmitate, 3 g high-purity egg yolk lecithin (EPCS), 0.2 g DSPE-PEG2000, 0.01 g citric acid and 3 g absolute ethanol. The mixture was dissolved by heating at 65° C. 75 g water for injection was heated to 50° C. to obtain a water phase. The organic phase was injected into the water phase under stirring conditions to obtain crude liposomes, which were homogenized and emulsified solution by using a high pressure homogenizer to obtain liposome. 10 g saccharose and 5 mannitol were dissolved in the liposome solution by stirring and diluted to 100 ml with water for injection. The pH value was adjusted to 7.0 with natrium hydroxydatum. The liposome was filtrated and sterilized through a 0.22 lam nylon syringe filter. Then, the filtrate was separately packaged, freeze-dried and cap-sealed to obtain docetaxel palmitate liposome solution with a mean particle size of 60.7 nm.

### Example 6

### Preparation of Docetaxel Palmitate Liposomes

[06] The organic phase was prepared with the prescription amount of 0.7 g docetaxel palmitate, 6 g high-purity egg yolk lecithin (EPCS), 0.7 g DSPE-PEG2000, 0.5 g cholesterol, 0.5 g citric acid and 6 g absolute ethanol. The mixture was dissolved by heating at 55° C. 80 g water for injection was heated to 55° C. to obtain a water phase. The organic phase was injected into the water phase under stirring conditions to obtain crude liposomes, which were homogenized and emulsified by using a high-pressure homogenizer to obtain liposome solution. The obtained liposome solution was diluted to 100 ml with water for injection. The pH value was adjusted to 4.80 with natrium hydroxydatum. The liposome was filtrated and sterilized through a 0.22 μm nylon syringe filter. Then, the filtrate was separately packaged, and cap-sealed to obtain docetaxel palmitate liposome solution with a mean particle size of 80.4 nm.

### Example 7

### Preparation of Docetaxel Palmitate Liposomes

[07] The organic phase was prepared with the prescription amount of 0.7 g docetaxel palmitate 2 g egg yolk lecithin, 1 g hydrogenated soy lecithin (HSPC), 0.5 g DSPE-PEG2000, 0.1 g cholesterol and 4 g absolute ethanol. The mixture was dissolved by heating at 55° C. 0.2 g trisodium citrate, 10 g trehalose, 12 g mannitol, 8 g glucose and 90 g water for injection were mixed and heated to 55° C. to obtain an aqueous phase. The organic phase was injected into the water phase under stirring conditions to obtain crude liposomes, which were separated via extruded membranes with a pore diameter of 0.8 μm, 0.4 μm and 0.2 μm to obtain liposome solution. The obtained liposome solution was diluted to 100 ml with water for injection. The pH value was adjusted to 4.50 with hydrochloric acid regulator. The liposome was filtrated and sterilized through a 0.22 μm nylon syringe filter. Then, the filtrate was separately packaged, freeze-dried and cap-sealed to obtain a liposomal docetaxel palmitate freeze-dried powder with a mean particle size of 150.0 nm.

### Example 8

### Preparation of Docetaxel Palmitate Liposomes

[08] The organic phase was prepared with the prescription amount of 0.8 g docetaxel palmitate, 3 g dipalmitoylphosphatidylcholine (DPPC), 3 g phosphatidylcholine, 1 g egg yolk lecithin, 0.8 g DSPE-PEG2000 and 8 g propylene glycol. The mixture was dissolved by heating at 70° C. 80 g water for injection was heated to 55° C. to obtain a water phase. The organic phase was injected into the water phase under stirring conditions to obtain crude liposomes, which were separated through the extruded membranes with a pore diameter of 0.8 μm, 0.4 μm, 0.2 μm and 0.1 μm to obtain liposome solution and propylene glycol was removed by ultrafiltration. 0.8 g trisodium citrate was placed in the liposome solution after ultrafiltration, stirred thoroughly and then diluted to 100 ml with water for injection. The pH value was adjusted to 9.0 with hydrochloric acid. The liposome was filtrated and sterilized through a 0.22 μm nylon syringe filter, and the filtrate was separately packaged and cap-sealed to obtain docetaxel palmitate liposome solution with a mean particle size of 145.2 nm.

### Example 9

### Preparation of Docetaxel Palmitate Liposomes

[09] The organic phase was prepared with the prescription amount of 0.1 gdocetaxel palmitate, 2 g soy lecithin, 0.1 g DSPE-PEG2000 and 6 g absolute ethanol. The mixture was stirred to dissolve by heating at 25° C. 0.5 g trisodium diaminetetraacetic acid, 0.5 g disodium citrate and 80 g water for injection were heated at 25° C. and stirred thoroughly to obtain the water phase. The organic phase was injected into the water phase under stirring conditions to obtain crude liposomes. The crude liposome was separated through extruded membranes with a pore diameter of 0.6 μm, 0.2 μm, 0.1 μm and 0.05 μm to obtain liposome solution, which was then diluted to 100 ml with water for injection. The pH value was adjusted to 3.50 with hydrochloric acid. The liposome was filtrated and sterilized through a 0.22 μm nylon syringe filter, and the filtrate was separately packaged and cap-sealed to obtain docetaxel palmitate liposome solution with a mean particle size of 118.8 nm.

### Example 10

### Preparation of Docetaxel Palmitate Liposomes

[10] The organic phase was prepared with the prescription amount of 1.0 g docetaxel palmitate, 10 g high-purity egg yolk lecithin (EPCS), 1.0 g DSPE-PEG2000, 1 g cholesterol and 10 g absolute ethanol. The mixture was heated at 60° C. while stirring. 74 g water for injection was heated at 60° C. to obtain the water phase. The organic phase was injected into the water phase under stirring conditions to obtain crude liposomes, which were homogenized and emulsified by using a high pressure homogenizer to obtain liposome solution, and absolute ethyl alcohol was removed by ultrafiltration. 0.5 g natrium lacticum and 0.5 g natrium malicum were placed in the liposome solution after ultrafiltration, stirred thoroughly and then diluted to 100 ml with water for injection. The pH value was adjusted to 5.0 with hydrochloric acid. The liposome was filtrated and sterilized through a 0.22 μm nylon syringe filter, and the filtrate was separately packaged and cap-sealed to obtain docetaxel palmitate liposome solution with a mean particle size of 130.2 nm.

### Example 11

### Preparation of Docetaxel Palmitate Liposomes

[11] The organic phase was prepared with the prescription amount of 0.1 g docetaxel palmitate, 1 g distearoylphosphatidylcholine, 0.05 g DSPE-PEG2000 and 1 g absolute ethanol. The mixture was heated at 55° C. while stirring. 95 g water for injection was heated at 55° C. to obtain the aqueous phase. The organic phase was injected into the water phase under stirring conditions to obtain crude liposomes. The crude liposome was homogenized and emulsified by using a high pressure homogenizer to obtain liposome solution. 0.001 g natrium lacticum was placed in the liposome solution, stirred thoroughly and then diluted to 100 ml with water for injection. The pH value was adjusted to 8.0 with natrium hydroxydatum. The liposome was filtrated and sterilized through a 0.22 μm nylon syringe filter, and the filtrate was separately packaged, freeze-dried and cap-sealed to obtain a liposomal docetaxel palmitate freeze-dried powder with a mean particle size of 90.7 nm.

### Example 12

### Preparation of Docetaxel Palmitate Liposomes

[12] The organic phase was prepared with the prescription amount of 0.1 g docetaxel palmitate, 1 g phosphatidylethanolamine, 1 g dimyristoylphosphatidylcholine, 0.5 g DSPE-PEG2000, 0.005 g ethylenediaminetetraacetic acid and 4 g absolute ethanol. The mixture was dissolved by heating at 55° C. 5 g trehalose was put in 70 g water for injection and heated at 55° C. The mixture was dissolved by stirring to obtain an aqueous phase. The organic phase was injected into the water phase under stirring conditions to obtain crude liposomes, which were then placed in an extruder and separated through extruded membranes with a pore diameter of 0.8 μm, 0.6 μm, 0.4 μm and 0.1 μm to obtain liposome solution. The obtained solution was diluted to 100 ml with water for injection. The pH value was adjusted to 7.5 with sodium hydroxide. The liposome was filtrated and sterilized through a 0.22 μm nylon syringe filter, and the filtrate was separately packaged, freeze-dried and cap-sealed to obtain a liposomal docetaxel palmitate freeze-dried powder with a mean particle size of 104.3 nm.

### Example 13

### Preparation of Docetaxel Palmitate Liposomes

[13] The organic phase was prepared with the prescription amount of 0.2 g docetaxel palmitate, 1 g phosphatidylserine, 1 g sphingomyelin, 0.2 g DSPE-PEG2000 and 0.01 g citric acid in a mixed solvent of 2 g absolute ethanol and 4 g propylene glycol. 50 g water was heated to 70° C. to obtain a water phase. The organic phase was injected into the water phase under stirring conditions to obtain crude liposomes, which were then homogenized and emulsified with a high-pressure homogenizer to obtain a liposome solution. 15 g sucrose, 15 g mannitol, 5 g erythritol and 5 g threonine were dissolved in the liposome solution by stirring and then diluted to 100 ml with water for injection. The pH value was adjusted to 6.0 with sodium hydroxide. The liposome was filtrated and sterilized through a 0.22 μm nylon syringe filter, and the filtrate was separately packaged, freeze-dried and cap-sealed to obtain a liposomal docetaxel palmitate freeze-dried powder with a mean particle size of 78.4 nm.

### Example 14

### Preparation of Docetaxel Palmitate Liposomes

[14] The organic phase was prepared with the prescription amount of 0.4 g docetaxel palmitate, 5 g high-purity egg yolk lecithin (EPCS), 0.5 g DSPE-PEG2000 and 0.01 g citric acid. The mixture was dissolved by heating at 50° C. 50 g water was heated to 50° C. to obtain a water phase. The organic phase was injected into the water phase under stirring conditions to obtain crude liposomes, which were then homogenized and emulsified with a high-pressure homogenizer, placed in an extruder, and separated through extruded membranes with a pore diameter of 0.2 μm, 0.1 μm, and 0.05 μm to obtain liposome solution. 10 g xylitol, 15 g sorbitol and 10 g mannitol were dissolved in the liposome solution by stirring and diluted to 100 ml with water for injection. The pH value was adjusted to 6.0 with sodium hydroxide. The liposome was filtrated and sterilized through a 0.22 μm nylon syringe filter and the filtrate was separately packaged, freeze-dried and cap-sealed to obtain a liposomal docetaxel palmitate freeze-dried powder with a mean particle size of 140.7 nm.

### Example 15

### Preparation of Docetaxel Palmitate Liposomes

[15] The organic phase was prepared with the prescription amount of 0.3 g docetaxel palmitate, 3 g high-purity egg yolk lecithin (EPCS), 0.1 g DSPE-PEG2000 and 5 g tert-butanol. The mixture was dissolved by heating at 45° C. 80 g water was heated to 45° C. to obtain a water phase. The organic phase was injected into the water phase under stirring conditions to obtain crude liposomes, which were then homogenized and emulsified with a high-pressure homogenizer to obtain liposome solution. 0.1 g trisodium citrate, 13 g sucrose and 5 g mannitol were dissolved in the liposome solution by stirring and then diluted to 100 ml with water for injection. The pH value was adjusted to 5.5 with hydrochloric acid. The liposome was filtrated and sterilized through a 0.22 μm nylon syringe filter, and the filtrate was separately packaged, freeze-dried and cap-sealed to obtain a liposomal docetaxel palmitate freeze-dried powder injection with a mean particle size of 105.3 nm.

### Example 16

### Preparation of Docetaxel Palmitate Liposomes

[16] The organic phase was prepared with the prescription amount of 0.3 g docetaxel palmitate, high purity egg yolk lecithin (EPCS) 1.5 g, hydrogenated soybean lecithin (HSPC) 0.5 g, 0.1 g DSPE-PEG2000 and 6 g absolute ethanol. The mixture was dissolved by heating at 55° C. 60 g water was heated to 55° C. to obtain a water phase. The organic phase was injected into the water phase under stirring conditions to obtain crude liposomes, which were then homogenized and emulsified with a high-pressure homogenizer to obtain liposome solution, with absolute ethanol removed by ultrafiltration. 0.3 g disodium edetate, 19 g trehalose and 5 g lactose were then dissolved in the liposome solution by stirring and diluted to 100 ml with water for injection. The pH value was adjusted to 7.0 with sodium hydroxide. The liposome was filtrated and sterilized through a 0.22 μm nylon syringe filter, and the filtrate was separately packaged, freeze-dried and cap-sealed to obtain a liposomal docetaxel palmitate freeze-dried powder injection with a mean particle size of 89.4 nm.

### Example 17

### Preparation of Docetaxel Palmitate Liposomes

[17] The organic phase was prepared with the prescription amount of 0.3 g docetaxel palmitate lipid, 3 g high-purity egg yolk lecithin (EPCS), 0.7 g DSPE-PEG2000, 0.1 g citric acid and 4 g propylene glycol. The mixture was dissolved by heating at 70° C. 10 g sucrose and 5 g trehalose were put in 70 g water for injection and heated at 70° C. The obtained mixture was dissolved by stirring to obtain an aqueous phase. The organic phase was injected into the water phase under stirring conditions to obtain crude liposomes, which were then placed in an extruder and separated through extruded membranes with a pore diameter of 0.8 μm, 0.6 μm, 0.4 μm, and 0.1 μm to obtain liposome solution. It was diluted to 100 ml with water for injection. The pH value was adjusted to 6.0 with sodium hydroxide. The liposome was filtrated and sterilized through a 0.22 μm nylon syringe filter. Then, the filtrate was separately packaged, freeze-dried and cap-sealed to obtain a liposomal docetaxel palmitate freeze-dried powder with a mean particle size of 113.6 nm.

### Example 18

### Preparation of Docetaxel Palmitate Liposomes

[18] Organic phase was prepared with the prescription amount of docetaxel palmitate 0.3 g, 3 g high-purity egg yolk lecithin (EPCS), 0.5 g DSPE-PEG2000, 0.01 g citric acid and 4 g propylene glycol. The mixture was dissolved by heating at 60° C. 17 g sucrose, 5 g mannitol was put in 65 g water for injection then heated at 70° C., in which the mixture was dissolved by stilling to obtain an aqueous phase. The organic phase was injected into the water phase under stirring conditions to obtain crude liposomes. The crude liposome was placed in an extruder and separated through extruded membranes with a pore diameter of 0.6 μm, 0.4 μm, 0.1 μm, and 0.05 μm to obtain liposome solution. The obtained solution was then diluted to 100 ml with water for injection. The pH value was adjusted to 6.7 with sodium hydroxide. The liposome was filtrated and sterilized through a 0.22 μm nylon syringe filter, and the obtained filtrate was separately packaged, freeze-dried and cap-sealed to obtain a liposomal docetaxel palmitate freeze-dried powder with a mean particle size of 106.3 nm.

### Example 19

### Effect of Chelating Agent on Anti-Tumor Effect of Docetaxel Palmitate Liposome

[19] The chelating agent contained in the prescription was the key to the substantial effect of the docetaxel palmitate liposome of the present invention. In order to further verify the superiority of the chelating agent in the present invention, multiple parallel comparisons were used to preparate liposomes containing the chelating agent and chelating agent-free liposomes under the same processing conditions, using mouse S180 sarcoma as a tumor model. The anti-tumor effects were compared between docetaxel palmitate liposomes containing and those without the chelating agent. The experimental design and results were shown below.

### 1. Sample Source

The commercially available docetaxel injection was used as a positive control drug, and the chelating agent-containing docetaxel palmitate liposome prepared in Example 1 was used as the test preparation. By strictly following the prescription of Example 1 in the process, liposomes without the chelating agent were prepared in parallel as control.

### 2. Establishment of the Mouse S180 Tumor Model and Design of the Dosing Regimen

Mouse ascites tumor S180 cells were cultured in DMEM medium at 37° C. and 5% CO₂, and passaged at a mean interval of 2 days. When cells grew to the logarithmic growth phase, they were injected into the abdominal cavity of the mice under aseptic conditions at an adjusted concentration of 5×10⁷ cells/mL. When obvious ascites was observed in about a week, ascites was drawn from the tumor-bearing mice aseptically and diluted with NS at an appropriate ratio of 1:5. The diluted ascites (0.2 mL) was inoculated into the mouse abdominal cavity. When the second-generation ascites was visible in about a week, it was drawn from the tumor-bearing mice aseptically, diluted with NS at a 1:5 ratio and prepared into a S180 cell suspension, which was then injected into the left armpit of the mice subcutaneously, 0.2 mL per mouse.

24 h after inoculation, the ICR mice were weighed and randomly divided into four groups (n=8/group): a blank control group, a commercial docetaxel injection group, a chelating agent-free docetaxel group, and a chelating agent-containing docetaxel group. The mice in the three docetaxel groups received 10 mg/kg docetaxel-based injection via the tail vein each time, and the mice in the blank control group received 0.2 ml NS daily, for a total of four administrations. On the third day of drug withdrawal, the mice were sacrificed and weighed, and the tumors were removed and weighed to calculate the tumor inhibition rate using the following equation:

Tumor inhibition rate=(tumor weight in NS group-tumor weight in the drug administration group)/tumor weight in NS group×100%

### 3. The Anti-Tumor Effect

Using mouse S180 sarcoma as a model, the anti-tumor effects of docetaxel palmitate liposomes with and without the chelating agent and the commercial docetaxel injection were investigated. The results are shown in Table 1.

TABLE 1
Docetaxel palmitate liposomes with and without the
chelating agent and comparative results of the anti-
tumor effect of the commercial docetaxel injection

| | Mean tumor | Tumor |
|---|---|---|
| Group | weight (g) | inhibition rate |
| Blank control | 1.17 ± 0.49 | / |
| Commercial docetaxel injection | 0.44 ± 0.21 | 62.39% |
| Docetaxel palmitate liposomes without | 0.30 ± 0.11 | 74.36% |
| the chelating agent | | |
| Docetaxel palmitate liposomes with | 0.21 ± 0.06 | 82.05% |
| the chelating agent | | |

### Result Analysis:

1. The anti-tumor effect of docetaxel palmitate liposomes with and without the chelating agent was significantly better than that of the commercial docetaxel injection, indicating that docetaxel was successfully developed into a prodrug docetaxel. The anti-tumor effect was significantly improved after modification with cypalmitate liposome, which is an important aspect of the substantial effect of the present invention.

2. The anti-tumor effects of docetaxel palmitate liposomes with and without the chelating agent were compared in parallel. The results showed that the anti-tumor effect of the liposomes with the chelating agent was better than that of the liposomes without the chelating agent.

In conclusion, the anti-tumor effect of docetaxel palmitate liposomes containing the chelating agent is improved as compared with those without, which is the embodiment of the substantial effect and the core technical feature of the present invention.

### Example 20

### Effect of the Chelating Agent on Pharmacokinetics of Docetaxel Palmitate Liposomes In Vivo

### [20] 1. Sample Source

A commercially available docetaxel injection was used as the reference preparation, the docetaxel palmitic acid liposome containing the chelating agent prepared completely according to the preparation process described in Example 1 was used as the chelating agent-containing docetaxel palmitic acid liposome sample, and the docetaxel palmitic acid liposome without containing the chelating agent prepared completely according to the preparation process described in Example 1 was used as the chelating agent-free docetaxel palmitic acid liposome sample.

### 2. Pharmacokinetic Test Design

Eighteen SD male rats were equally randomized into three groups: a commercial docetaxel injection group, a group of docetaxel palmitic acid liposome without the chelating agent, and a group of docetaxel palmitic acid liposome containing the chelating agent. Before the experiment, the animals were fasted overnight with free access to drinking water, and then received 10 mg/kg docetaxel-based injection via the tail vein. At 0.033, 0.083, 0.167, 0.25, 0.5, 0.75, 1 1.5, 2, 3, 4, 6, 8, 10, 12 and 24 h after drug administration, 0.5 mL blood was drawn from the orbital venous plexus, placed in a centrifuge tube containing heparin sodium, shaken well, and centrifuged at 4500 rpm for 10 min. 150 μl plasma was taken, stored at −20° C., and processed according to the conventional method. The plasma concentration of docetaxel was determined by high performance liquid chromatography (HPLC).

### 3. Results and Analysis

DAS 2.0 software was used for model fitting, and the pharmacokinetic parameters were calculated. The in vivo pharmacokinetic results of the commercial docetaxel injection, chelating agent-containing docetaxel palmitate liposomes and chelating agent-free docetaxel palmitate liposomes are shown in Table 2.

TABLE 2
Comparison of the main pharmacokinetic parameters between
the commercial docetaxel injection group, chelating agent-
containing docetaxel palmitate liposome group and chelating
agent-free docetaxel palmitate liposome group

| | | | Docetaxel | Docetaxel |
|---|---|---|---|---|
| | | | palmitate | palmitate |
| | | Commercial | liposome | liposome |
| | | docetaxel | without the | with the |
| Index | Unit | injection | chelating agent | chelating agent |
| AUC_{∞} | μg/mL*h | 4.729 | 18.210 | 22.112 |
| Cₘₐₓ | μg/mL | 6.921 | 3.061 | 2.824 |
| T_{1/2} | h | 5.902 | 9.315 | 10.425 |
| CL | L/h/kg | 2.115 | 0.549 | 0.494 |
| MRT | h | 1.232 | 3.744 | 4.891 |

### Result Analysis:

1. AUCs and T½ of docetaxel palmitate liposomes were significantly larger than those of the commercially available docetaxel palmitate injections. The results of this experiment demonstrated that the docetaxel into prodrug liposome preparation was able to delay the metabolism of the drug in the body and prolong the action time remarkably.

2. Compared with docetaxel palmitate liposomes without chelating agents, AUC(x) was increased and T½ was prolonged in docetaxel palmitate liposomes containing the chelating agent. From the perspective of the pharmacological effect, liposomes containing the chelating agent had a better anti-tumor effect, perhaps because they have a relatively longer action time in the body.

In conclusion, after addition of the chelating agent to the formulation of docetaxel palmitate liposomes, the in vivo action time of the drug was prolonged and the anti-tumor effect was improved, indicating that the chelating agent in the prescription plays a particularly important role in the docetaxel palmitate liposome of the present invention and is the key technical feature of the prevent invention.

### Example 21

### Effect of the Chelating Agent on the Characteristics of the Docetaxel Palmitate Liposome Formulation

[21] As the research object of the present invention is a kind of liposome which cannot be sterilized at high temperature during the production process, sterilization is usually affected by filtration via a 0.22 μm filter membrane. In the actual production process, the large liposome particle size or uneven PDI often results in poor sterilization and filtration, which seriously affects the production efficiency. For this reason, we paid special attention to the smoothness of filtration and sterilization of the docetaxel palmitate liposomes during the research process and found that addition of the chelating agent could make the filtration process more smooth, because both the particle size and PDI of the liposomes with the chelating agent are slightly smaller than those without the chelating agent. The experimental design and results are shown below.

In Example 1 for instance, 1000 ml docetaxel palmitate liquid liposomes with and without the chelating agent were prepared completely according to the recipe described in Example 1 by using an 11 mm plate filter and a 0.22 μm polyethersulfone membrane. The filtration volume was recorded. The particle size and PDS of the docetaxel palmitate liquid liposomes with and without the chelating agent were measured and the results are shown in Table 3

TABLE 3
Comparison of the characteristics of the docetaxel palmitate
liquid liposomes with and without the chelating agent

| | | |
|---|---|---|
| | Docetaxel palmitate | Docetaxel palmitate |
| | liposomes without | liposomes with |
| Index | the chelating agent | the chelating agent |

| | | | | |
|---|---|---|---|---|
| Mean particle size | 102.1 | nm | 92.4 | nm |

| | | |
|---|---|---|
| PDI | 0.237 | 0.124 |

| | | | | |
|---|---|---|---|---|
| Filter volume | 625 | ml | 867 | ml |

### Result Analysis:

The docetaxel palmitate liposome of the present invention has smaller particle size, narrower distribution, and smoother sterilization filtration. It can be seen that after addition of the chelating agent, the basic properties of the formulation are significantly improved and the production is implemented more smoothly, which further reflects the superiority of the chelating agent contained in the prescription.

We have described the preferred embodiment of the present invention in detail but the present invention is not limited to the embodiment described. Technicians and researchers who are familiar with the trade can make various equivalents as long as they do not violate the spirit of the present invention and these equivalent variations or replacements are all included in the scope defined by the claims of this application.

1. A docetaxel palmitate liposome, which contains docetaxel palmitate as the main drug, a chelating agent, lecithin and DSPE-PEG2000, with a respective content of 0.1-2%, 0.001-1%, 1-10% and 0.05-1%.

2. The docetaxel palmitate liposome according to claim 1, wherein the liposome is a freeze-dried powder injection.

3. The docetaxel palmitate liposome according to claim 1, wherein the liposome is a liposome solution for injection.

4. The docetaxel palmitate liposome according to claim 1, which is specifically formulated by the following:

| | | |
|---|---|---|
| Docetaxel palmitate | 0.1-1% | g/mL; |
| Lecithin | 1-10% | g/mL; |
| DSPE-PEG2000 | 0.05-1.0% | g/mL; |
| Cholesterol | 0~1% | g/mL; |
| Chelating agent | 0.001-1% | g/mL; |
| Lyophilized protective agent | 0~40% | g/mL; |

pH is adjusted to 3.5~9.0 using a pH adjuster; and the remaining is the water for injection.

5. The docetaxel palmitate liposome according to claim 1, which is specifically formulated by the following:

| | | |
|---|---|---|
| Docetaxel palmitate | 0.1-0.8% | g/mL; |
| Lecithin | 2-7% | g/mL; |
| DSPE-PEG2000 | 0.1-0.8% | g/mL; |
| Cholesterol | 0~0.6% | g/mL; |
| Chelating agent | 0.005-0.8% | g/mL; |
| Lyophilized protective agent | 5~35% | g/mL; |

pH is adjusted to 3.5~8.0 using a pH adjuster; and the remaining is the water for injection.

6. The docetaxel palmitate liposome according to claim 1, which is specifically formulated by the following:

| | | |
|---|---|---|
| Docetaxel palmitate | 0.2-0.7% | g/mL; |
| Lecithin | 3-6% | g/mL; |
| DSPE-PEG2000 | 0.2-0.7% | g/mL; |
| Cholesterol | 0~0.5% | g/mL; |
| Chelating agent | 0.01-0.5% | g/mL; |
| Lyophilized protective agent | 10~30% | g/mL; |

pH is adjusted to 3.5~7.0 using a pH adjuster; and the remaining is the water for injection.

7. The docetaxel palmitate liposome according to claim 1, wherein the lecithin is selected from high purity egg yolk lecithin (EPCS), hydrogenated soybean lecithin (HSPC), dipalmitoylphosphatidylcholine (DPPC), phosphatidylcholine, egg yolk lecithin, soy lecithin, phosphatidylserine, dimyristoylphosphatidylcholine, distearoylphosphatidylcholine, phospholipids, one or more of acylethanolamine and sphingomyelin.

8. The docetaxel palmitate liposome according to claim 1, is characterized in that the chelating agent is selected from one or more of the following: citric acid, disodium citrate, trisodium citrate, lactic acid, sodium lactate, malic acid, sodium malate, ethylenediamine tetraacetic acid, disodium ethylenediamine tetraacetate and trisodium ethylenediamine tetraacetate.

9. The docetaxel palmitate liposome according to claim 4, wherein the freeze-dried protective agent is selected from one or more of the following:
trehalose, sucrose, maltose, lactose, mannitol, glucose, sorbitol, xylitol, erythritol, and threonine.

10. The docetaxel palmitate liposome according to claim 4, wherein the pH adjusting agent is one or more of sodium hydroxide and hydrochloric acid.

11. The docetaxel palmitate liposome according to claim 1, wherein the liposome has a particle size of 50-150 nm.

12. The preparation method of docetaxel palmitate liposomes according to claim 1, wherein the preparation method is as follows:
Weigh the prescription amount of docetaxel palmitate, cholesterol, phospholipid, DSPE-PEG2000, chelating agent, put them in an organic solvent for injection and dissolve them by heating at 25-70° C. to obtain the organic phase; heat a proper amount of water to 25-70° C. to obtain the water phase; pour the organic phase into the water phase under stirring and mix them well to obtain crude liposomes; emulsify the crude liposomes and place them under high pressure; perform homogenization and emulsification in a homogenizer, or place them in an extruder to extrude through extruded membranes with different pore diameters, or extrude after high-pressure homogenization to obtain a liposome solution; lyophilized protective agent, place it in the above liposome solution and dissolve it by stirring and dilute it to the full volume with water for injection; adjust the pH value with a pH adjuster; finally, sterilize, pack and seal it through a 0.22 μm filter membrane to obtain so-called liposomes of liposome docetaxel palmitate. A powder form of docetaxel palmitate liposomes can also be prepared by lyophilization.

13. The method for preparing docetaxel palmitate liposomes according to claim 12, wherein the organic solvent for injection is selected from one, two or more of the following: propylene glycol, absolute ethanol, and tert-butanol at a dose of 1-10% g/mL.

14. The method for preparing docetaxel palmitate liposomes according to claim 12, wherein the organic solvent for injection can be retained in liposomes or in crude liposomes. After emulsification, it can be removed by ultrafiltration, or freeze-drying.

15. The method for preparing docetaxel palmitate liposomes according to claim 12, characterized in that the crude liposomes are emulsified, and the pore size of the extruded membrane is selected from among 0.8 μm, 0.6 μm, 0.4 μm, 0.2 μm, 0.1 μm and 0.05 μm by one, two or more in turn through extrusion of large pores to small pores.

16. The method for preparing docetaxel palmitate liposomes according to claim 12, wherein the chelating agent is dissolved in an oil phase, an aqueous phase or a liposome solution.

17. The method for preparing docetaxel palmitate liposomes according to claim 12, wherein the lyophilized protective agent is dissolved in a liposome solution, or an aqueous phase.

18. The docetaxel palmitate liposome according to claim 2, which is specifically formulated by the following:

| | | |
|---|---|---|
| Docetaxel palmitate | 0.1-1% | g/mL; |
| Lecithin | 1-10% | g/mL; |
| DSPE-PEG2000 | 0.05-1.0% | g/mL; |
| Cholesterol | 0~1% | g/mL; |
| Chelating agent | 0.001-1% | g/mL; |
| Lyophilized protective agent | 0~40% | g/mL; |

pH is adjusted to 3.5~9.0 using a pH adjuster; and the remaining is the water for injection.

19. The docetaxel palmitate liposome according to claim 3, which is specifically formulated by the following:

| | | |
|---|---|---|
| Docetaxel palmitate | 0.1-1% | g/mL; |
| Lecithin | 1-10% | g/mL; |
| DSPE-PEG2000 | 0.05-1.0% | g/mL; |
| Cholesterol | 0~1% | g/mL; |
| Chelating agent | 0.001-1% | g/mL; |
| Lyophilized protective agent | 0~40% | g/mL; |

pH is adjusted to 3.5~9.0 using a pH adjuster; and the remaining is the water for injection.

20. The docetaxel palmitate liposome according to claim 2, which is specifically formulated by the following:

| | | |
|---|---|---|
| Docetaxel palmitate | 0.1-0.8% | g/mL; |
| Lecithin | 2-7% | g/mL; |
| DSPE-PEG2000 | 0.1-0.8% | g/mL; |
| Cholesterol | 0~0.6% | g/mL; |
| Chelating agent | 0.005-0.8% | g/mL; |
| Lyophilized protective agent | 5~35% | g/mL; |

pH is adjusted to 3.5~9.0 using a pH adjuster; and the remaining is the water for injection.
